(19) United States
(12) Patent Application Publication
(10) Pub. No.: US 2025/0378948 A1
(43) Pub. Date: Dec. 11, 2025

(54) AI-BASED PERSONALIZED MEDICAL INFORMATION PROVISION SYSTEM AND METHOD THEREOF

(71) Applicant: Myung Jae LIM, Ansan-si (KR)

(72) Inventor: Myung Jae LIM, Ansan-si (KR)

(21) Appl. No.: 19/302,289

(22) Filed: Aug. 18, 2025

Related U.S. Application Data
(63) Continuation-in-part of application No. 18/089,593, filed on Dec. 28, 2022, now abandoned.

(51) Int. Cl. G16H 40/67; G06Q 30/0207; G16H 20/10; G16H 50/70

(52) U.S. Cl. CPC G16H 40/67; G06Q 30/0224; G16H 20/10; G16H 50/70; G06Q 2220/00

(57) ABSTRACT

A system for providing AI-based personalized medical information according to an embodiment of the present disclosure includes a user terminal configured to receive prescription information from a hospital terminal through an Open API, a pharmacy terminal configured to transmit data by generating drug guidance information based on the prescription information, and an AI medical information consulting server that is configured to evaluate a disease type, a disease severity, and a physical safety by analyzing the drug type, dosage, administration frequency, and prescription period stored in the drug guidance information provided in the Open API of the user terminal, and to provide personalized healthcare content for users based on the analysis results.

### CROSS-REFERENCE TO RELATED APPLICATIONS

This application is a continuation-in-part of U.S. application Ser. No. 18/089,593, filed Dec. 28, 2022 in the U.S. Patent and Trademark Office. All disclosures of the document named above are incorporated herein by reference.

### TECHNICAL FIELD

The present disclosure relates to a system and method for providing customized medical information based on artificial intelligence, and more particularly, a system and method in which AI technology is used to assign a certain code or number to drug specified in a prescription issued by a user and provide a list of nearby pharmacies based on user's location information, transmit prescription information to the terminal of the pharmacy selected by the patient so that prescription drugs can be prepared, provide customized drug including drug guidance video or health management video, additional information related to a disease to a user terminal and provide advertisements of medical institutions related to the customized medical information.

### DISCUSSION OF RELATED ART

Generally, patients receive a prescription after treatment is finished, go to a pharmacy, present the prescription and purchase the prescribed drug. However, if patients have an underlying disease or a rare condition, or if they need to take multiple drugs in combination, a doctor or pharmacist will explain specific drug-taking instructions, but the contents are complicated. Hence, patients forget how to take the drug or follow the wrong instructions. In addition, it is a reality that it is difficult for patients to acquire sufficient information related to their disease or listen to explanations due to the limited doctor's consultation hours.

In addition, due to the era of super-aging, patients have a problem in that they have to take multiple drugs simultaneously as the number of drugs they need to take increases. Moreover, these drugs may differ in the timing of taking them or how they work, and there is more information to be aware of. Further, it is often difficult for elderly patients to understand simple oral drugs, but also insulin injections, inhalation devices, eye drops, and adhesive drugs, as well as auditorial explanations or written guidance when the patients have to operate and take them at home alone. Many problems arise when elderly patients acquire, understand, and store this information, as this information is explained orally by medical personnel or provided only in writing. In particular, as paper documents are provided, environmental destruction and waste of money occur.

However, producing a video of numerous drugs and health information is costly and takes a lot of time in order to address the above issues.

### SUMMARY

An object of the embodiments of the present disclosure is to provide customized medical information based on patient's prescription information.

Another object of the embodiments of the present disclosure is to provide an accurate understanding of the patient's own disease and prescription drugs to improve public health and reduce environmental waste by replacing paper documents at the same time. Another object of the embodiments of the present disclosure is to motivate experts in the medical field to voluntarily create customized medical information contents.

A system for providing AI-based personalized medical information according to one embodiment of the present disclosure, comprising at least one processor and a memory, comprising a user terminal configured to receive prescription information from a hospital terminal through an Open API, a pharmacy terminal configured to generate drug guidance information based on the prescription data and to transmit the drug guidance information to the user terminal, and an AI medical information consulting server comprising the processor and the memory storing instructions that, when executed by the processor, cause the server to operate: a drug data extraction module configured to extract structured drug data, including a drug type, dosage, administration frequency and prescription period from the drug guidance information; a data processing module configured to perform preprocessing and feature extraction on the structured drug data; an inference module configured to apply a pre-trained machine learning model stored in the memory to the preprocessed and feature-extracted drug data to infer a disease type, a disease severity, and a physical safety status of the user a content generation module configured to generate personalized healthcare content including alerts, recommendations, and health tracking prompts based on the inference result; and a content provision module configured to transmit the generated personalized healthcare content to the user terminal in real time through a secure communication channel; wherein the machine learning model is the trained based on training dataset, including past prescription data, health profiles of a user, and treatment outcomes, and is continuously updated based on user feedback or user feedback.

In other embodiments, the content provision module of AI medical information consulting server searchs a plurality of pre-stored drug guidance video contents, and selects content corresponding to an age group or gender of the user to transmit the selected content to the user terminal via an Open API or social networking service SNS.

In other embodiments, the inference module of the AI medical information consulting server evaluates a severity level of a disease based on at least one of a drug type, dosage, administration frequency, and prescription period, and analyzes whether the user has a comorbidity or complications based on user physical information and information regarding concurrently administered drugs through a rule-based inference engine.

In other embodiments, the inference module of the AI medical information consulting server analyzes a user's past prescription history in chronological order, and evaluates whether the user's health condition has improved, deteriorated, or remained stable using a time series analysis.

In other embodiments, the inference module of the AI medical information consulting server monitors a viewing history of video content played on the user terminal, and runs a behavioral analysis algorithm that measures the treatment adherence level by comparing the monitored viewing frequency with an average viewing frequency of other users.

In other embodiments, the content provision module of the AI medical information consulting server provides video content to users by adjusting an exposure frequency of warning video content based on the evaluation of treatment adherence.

In other embodiments, the AI medical information consulting server further includes the user engagement score management module that monitors the participation level of users by analyzing dietary patterns and exercise records received from the user terminal, and generates the user engagement scores by comparing the participation level of the user with those of other users within the user cluster based on age and gender.

In other embodiments, the user engagement score management module calculates accumulated points based on at least one of the user's content viewing frequency, the number of blood glucose record entries, the number of dietary record entries, and the number of exercise record entries, and stores the accumulated points in a point management table for each user.

In other embodiments, the user engagement score management module applies rankings depending on the user engagement scores within the user cluster, and calculates real-time grade of the user based on the degree of disease improvement, the level of health achievements, and the duration of health maintenance.

In other embodiments, the user engagement score management module adjusts the user's real-time ranking based on a change history of the user's prescription information, including at least one of dosage, administration frequency, and prescription period.

In other embodiments, the user engagement score management module provides the user interface UI for preparing health-related know-how content to the user terminal having a grade equal to or higher than a predetermined threshold; assigns the pre-defined reward points based on the viewing frequency of video content created by the corresponding user terminal; and records a transaction history of the reward points in blockchain-based storage.

Meanwhile, a method of providing AI-based personalized medical information to a user according to an embodiment of the present disclosure comprises: receiving, by a user terminal via an Open API, prescription information from a hospital terminal; generating, by a pharmacy terminal, drug guidance information based on the prescription information and transmitting the drug guidance information to the user terminal; extracting, by a drug data extraction module of the AI medical information consulting server, structured drug data including a drug type, dosage, administration frequency and prescription period from the drug guidance information; performing, by a data processing module of the AI medical information consulting server, preprocessing and feature extraction on the structured drug data; inferring, by an inference module of the AI medical information consulting server, a disease type, disease severity, and physical safety status of the user by applying a pre-trained machine learning model stored in the memory to the preprocessed and feature-extracted drug data; generating, by a content generation module of the AI medical information consulting server, personalized healthcare content including alerts, recommendations, and health tracking prompts based on the inference result; and transmitting, by a content provision module of the AI medical information consulting server, the generated personalized healthcare content to the user terminal in real time through a secure communication channel, wherein the machine learning model is trained based on a training dataset including past prescription data, user health profiles, and treatment outcomes, and is continuously updated based on user feedback or result verification.

In other embodiments, the step of transmitting the generated personalized healthcare content to the user terminal in real time comprises: searching a plurality of pre-stored medication guidance video contents; selecting content corresponding to an age group or gender of the user; and transmitting the selected content to the user terminal via an SNS or an Open API.

In other embodiments, the step of inferring the user's disease type, disease severity, and physical safety status comprises: evaluating the severity of the disease according to a drug type, dosage, administration frequency and prescription period; and analyzing, through a rule-based inference engine, whether the user has a comorbid condition or complication based on the user's physical information and information on concurrently administered medications.

In other embodiments, the step of inferring the user's disease type, disease severity, and physical safety status comprises: analyzing the user's past prescription history in chronological order; and evaluating, through a time-series analysis model, whether the user's health condition has improved, deteriorated, or remained stable.

In other embodiments, the step of inferring the user's disease type, disease severity, and physical safety status comprises: monitoring the viewing history of content executed on the user terminal; and evaluating, through a behavioral analysis algorithm, the user's treatment adherence by comparing the viewing frequency with an average viewing frequency of other users.

In other embodiments, the method of providing AI-based personalized medical information to a user further comprises: analyzing exercise records and dietary improvement records received from the user terminal; monitoring the user's participation level; and generating a user participation score by comparing the user's participation level within a user cluster based on age and gender.

According to one embodiment of the present disclosure, it connects the hospital terminal, pharmacy terminal, and user terminal to infer a disease type, a disease severity, and a physical safety status of the user based on prescription information, and the personalized healthcare consulting services can be offered based on this.

In addition, according to one embodiment, structured drug data may be extracted, and a machine learning model may be applied through preprocessing and feature extraction, so that the user's health condition can be monitored and evaluated in real time based on medication guidance information.

In addition, according to one embodiment, it evaluates the user's long-term health status using a time series analysis model and past prescription history to offer effective data analysis and a precise understanding of the user's disease and prescription and also support the users to actively take suitable actions for their own health.

In addition, according to one embodiment, it monitors the participation level of users based on the number of exercise records, dietary pattern records, and content viewing frequency to evaluate, and support the continuous improvement of health management based on the user participation level.

Furthermore, according to one embodiment, it calculates points according to users' activities, and applies rankings and points depending on the user engagement scores in the user cluster to increase a user's voluntary participation and attention for health care.

In addition, according to one embodiment, it promotes users' participation, such as allowing users to personally prepare health-related know-how content and awarding points according to the number of content viewing frequency.

### BRIEF DESCRIPTION OF THE DRAWINGS

FIG. 1 is a configuration diagram schematically illustrating an artificial intelligence-based customized medical information provision system according to an embodiment of the present disclosure.

FIG. 2 is a configuration diagram schematically illustrating a medical information provision server according to an embodiment of the present disclosure.

FIG. 3 is a flowchart illustrating an artificial intelligence-based customized medical information provision method according to an embodiment of the present disclosure.

FIG. 4 is a flowchart illustrating a preparation process for generating customized medical information according to an embodiment of the present disclosure.

FIG. 5 is a flowchart illustrating a pre-processing process for a drug guidance video or health management video for registration the video to a video sharing platform according to an embodiment of the present disclosure.

FIG. 6 is a configuration diagram schematically illustrating an AI-based personalized medical information provision system according to an embodiment of the present disclosure.

FIG. 7 is a configuration diagram, depicting details of AI medical information consulting server shown in FIG. 6.

FIG. 8 is an example diagram of the Open API functions running on the user terminal shown in FIG. 6.

FIG. 9 amd FIG. 10 are flowcharts illustrating a method for providing AI-based personalized medical information according to an embodiment of the present disclosure

### DETAILED DESCRIPTION OF EXEMPLARY EMBODIMENTS

Hereinafter, the present disclosure as described above is described in detail through the accompanying drawings and embodiments.

It should be noted that technical terms used in the present disclosure are only used to describe specific embodiments and are not intended to limit the present disclosure. Further, technical terms used in the present disclosure should be interpreted in terms commonly understood by those skilled in the art to which the present disclosure belongs, unless specifically defined otherwise in the present disclosure, and are excessively inclusive. It should not be interpreted in an excessively positive sense or in an excessively reduced sense. Further, when the technical terms used in the present disclosure are erroneous technical terms that do not accurately express the spirit of the present disclosure, they should be replaced with technical terms that those skilled in the art can correctly understand. Further, general terms should be interpreted as defined in the dictionary or according to context and should not be interpreted in an excessively reduced sense.

Further, singular expressions used in the present disclosure include plural expressions unless the context clearly indicates. Terms such as “consisting of” or “comprising” used in the present disclosure should not be construed as necessarily including all of the various components or steps described in the invention, and it should be construed that some components or steps among them may not be included, or additional components or steps may be further included.

Further, terms including ordinal numbers such as “first” and “second” used in the present disclosure may be used to describe components, but components should not be limited by the terms. Terms are used only to distinguish one component from another. For example, a first element may be termed a second element, and similarly, a second element may be termed a first element, without departing from the scope of the present disclosure.

The semantic meaning of “user” used in the present disclosure may be interpreted as having the same meaning as “patient.”

Hereinafter, preferred embodiments according to the present disclosure are described in detail with reference to the accompanying drawings. However, regardless of reference numerals, the same or similar components are given the same reference numerals, and overlapping descriptions thereof are excluded.

Further, in describing the present disclosure, if it is determined that a detailed description of a related known technology may obscure the gist of the present disclosure, the detailed description will be excluded. Further, it should be noted that the accompanying drawings are only for easily understanding the spirit of the present disclosure and should not be construed as limiting the spirit of the present disclosure by the accompanying drawings.

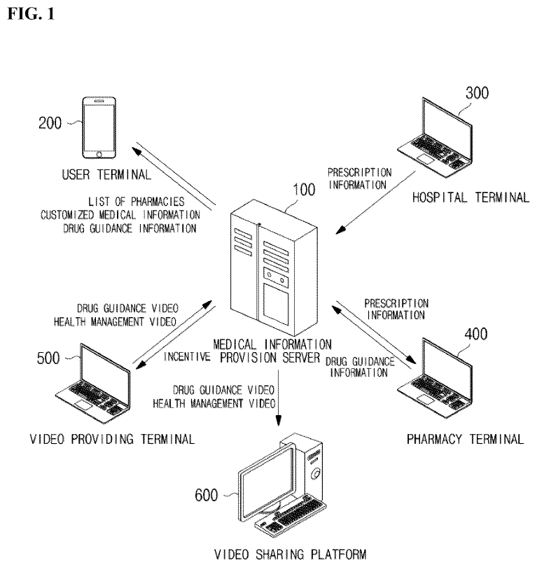

FIG. 1 is a configuration diagram schematically illustrating an artificial intelligence (AI)-based customized medical information provision system according to an embodiment of the present disclosure.

Referring to FIG. 1, the AI-based customized medical information provision system may comprise a medical information provision server 100, a user terminal 200, a hospital terminal 300, a pharmacy terminal 400, a video providing terminal 500, and a video sharing platform 600.

Each component of FIG. 1 is generally connected through a network. For example, the user terminal 200 may be connected to the medical information provision server 100, the pharmacy terminal 400, and the video sharing platform 600 through a network. The medical information provision server 100 may be connected to the user terminal 200, the hospital terminal 300, the pharmacy terminal 400, the video providing terminal 500, the video sharing platform 600, etc., through a network.

The network refers to a connection structure capable of exchanging information between nodes such as a plurality of terminals and servers, and examples of such networks include a local area network (LAN) and a wide area network (WAN), the Internet (WWW: World Wide Web), wired and wireless data communications networks, telephone networks, and wired and wireless television communications networks. Examples of wireless data communication networks include 3G, 4G, 5G, 3rd generation partnership project (3GPP), 5th generation partnership project (5GPP), long-term evolution (LTE), world interoperability for microwave access (WiMAX), Wi-Fi, Internet, local area network (LAN), wireless local area network (wireless LAN), wide area network (WAN), personal area network (PAN), radio frequency (RF), Bluetooth network, near-field communication (NFC) networks, satellite broadcasting networks, analog broadcasting networks, digital multimedia broadcasting (DMB) networks, etc., but are not limited thereto.

The user terminal 200 may transmit the prescription information received from the medical information providing server 100 to the pharmacy terminal 400 selected by the user. It may receive customized medical information from the medical information provision server 100 and display the customized medical information.

The prescription information is information input by a doctor to issue a prescription. This includes various information such as insurance classification, personal information, hospital information, disease classification code, the doctor in charge information, drug name, single dose, number of administrations per day, the total number of administration days, and usage. The prescription information input by a doctor in the hospital terminal 300 may be automatically transmitted to the medical information provision server 100.

According to an embodiment, the user terminal 200 may receive the prescription information through a customized medical information application provided by the medical information provision server 100.

Further, when a list of a plurality of nearby pharmacies based on the user's location information identified through a built-in GPS or mobile communication network is displayed in the customized medical information application of the user terminal 200, the user terminal 200 may transmit the prescription information according to the user's selection to the pharmacy terminal 400 corresponding to the selected pharmacy. Further, the prescription information may be obtained through optical character recognition (OCR) of an image of a prescription provided by a hospital by the user, or through QR code recognition provided in a prescription. This way, prescription information may be obtained through prescription information input from the hospital terminal 300 and OCR or QR code recognition of the user terminal 200.

According to an embodiment, when a screen displayed on a blood glucose meter or a blood pressure monitor is photographed through a camera (not shown) of the user terminal 200 and gender and age are input, if gender and age are input, the captured screen, gender, and age are analyzed to automatically provide in video with high relevance, thereby checking user's health condition and reviewing products or services related to user's disease through advertisements provided along with the video. Further, when a specific word is entered on the search screen of the customized medical information application, a list of videos related to the word is provided, and specialized video information may be provided to the user by selecting it.

The medical information provision server 100 may receive prescription information from the hospital terminal 300, transmit the prescription information to the user terminal 200 and transmit the customized medical information to the user terminal 200. More specifically, the medical information provision server 100 generates medical information, including at least one drug guidance video, health management image, or additional information related to a disease registered in the video-sharing platform 600 based on the prescription information. The medical information provision server 100 may provide a customized medical information application to the user terminal 200 and provide customized medical information through the customized medical information application installed in the user terminal 200.

The medical information provision server 100 provides prescription information, customized medical information, and a list of a plurality of nearby pharmacies based on location information of the user terminal 200 through a customized medical information application installed on the user terminal 200.

According to an embodiment, the medical information provision server 100 provides various customized medical information such as drug guidance video, health management video, and additional information related to a disease through a customized medical information application. However, it provides customized advertisements related to each piece of information may be of interest to the user.

The hospital terminal 300 may be a terminal into which prescription information is input by a doctor. The hospital terminal 300 may be configured to automatically transmit the prescription information to the medical information provision server 100 as soon as the prescription information is input.

According to an embodiment, the prescription information may be obtained through optical character recognition (OCR) of an image taken by the user terminal 200 of a prescription provided by a user from a hospital. Further, the prescription information may be obtained through QR code recognition by providing a QR code to the prescription. This way, prescription information may be directly obtained from the user terminal 200 and transmitted to the pharmacy terminal 400.

According to an embodiment, the hospital terminal 300 may request registration of advertisements for hospital information and medical products provided by the hospital to the medical information provision server 100. For example, it is possible to request registration of advertisements for health-functional foods, supplements, treatment-related medical devices, etc., helpful for the user's disease to the medical information provision server 100.

The pharmacy terminal 400 may be a terminal displaying prescription drug order information based on the prescription information received from the medical information provision server 100 and performing a payment function.

According to an embodiment, the pharmacy terminal 400 may request registration of advertisements for pharmacy information and various products and the like to the medical information provision server 100. For example, like the hospital terminal 300, information on health-functional foods or supplements suitable for the type of disease of the user identified through prescription information is produced as an advertisement, and the pharmacy terminal 400 may request registration of advertisements to the medical information provision server 100.

In this way, the medical information provision server 100 may receive advertisement requests from various institutions related to medical care, such as the hospital terminal 300, pharmacy terminal 400, insurance company, financial company, etc., and store them in a database (not shown), and provide advertisements highly related to a disease of a specific patient among stored advertisements to the user terminal 200. Here, the degree of relevance, which is a criterion for matching, may be based on keywords.

According to an embodiment, the pharmacy terminal 400 may transmit drug guidance information input based on the prescription information to the user terminal 200. In this case, the drug guidance information may be simply inputted by the pharmacist through the pharmacy terminal 400, such as used for prescription drugs. For example, drug guidance information in the form of voice or text from the pharmacist regarding 30 minutes after a meal, 30 minutes before a meal, 3 times a day, a list of foods to be careful of during the intake period, possible side effects and countermeasures may be provided. In this way, prescription information is transmitted to the selected pharmacy terminal based on the location of the user terminal 200 in advance so that without the user arriving at the pharmacy after treatment, presenting the prescription, selecting the pharmacy and waiting for prescription drug to be prepared, the desired pharmacy is selected and prescription information is transmitted immediately, time to pick up the prescription may be saved upon arrival at the pharmacy. Further, drug guidance information input from the pharmacist is received to check again at any time about usage or precautions, which helps to take medicine normally according to the usage.

The video providing terminal 500 may receive a combined serial number by assigning a unique number for each attribute of the drug and a unique number for each subject from the medical information provision server 100 and produce at least one drug guidance video or health management video corresponding to the serial number. The video providing terminal 500 may transmit at least one drug guidance video or health management video to the medical information provision server 100. To explain this in detail, Tylenol™ is given a unique number for each attribute such as 6 digits 112201 through antipyretic analgesic-11, acetaminophen-22, and 500 mg-01. Further, if the content is changed to 650 mg, although the efficacy and the same ingredient, the last two digits may be designated as 02. Further, additional unique numbers for gender and age are assigned to the above number so that a unique number for each attribute of drug and a unique number for each subject of drug may be given to store the combined serial number. For example, there are 112201-MO, 112201-FY, 112201-Y, and 112201-O in which serial numbers such as M=male, F=female, Y=young, and O=old may be assigned to each drug. Further, additional unique numbers may be assigned for underlying diseases, allergies, and the like.

According to one embodiment, preferably, students of medical school, pharmacy school, dental school, etc., which may be regarded as experts in the medical field, may provide drug guidance video or health management video through the video providing terminal 500 but are limited thereto. Those who can produce medical information-related videos may also provide drug guidance video or health management video. In this way, high-quality content may be supplied through cloud sourcing for the serial number list provided by the medical information provision unit. The serial number list may be displayed on the video providing the terminal 500 with the serial number and names, efficacy, ingredients, and contents, which are attributes of drugs corresponding to the serial numbers. In addition, video standard information including video time, quality, format, etc. may be additionally provided to secure video consistency. The obtained drug guidance video and health management video are evaluated to calculate an evaluation score. When the evaluation score is above a reference value, it is registered on a video-sharing platform, and an incentive may be paid to the video provider. For example, the evaluation of a drug guidance video or health management video may be performed by qualified holders of a specialist or higher in each department pre-registered in the medical information provision server 100, and a video with a score of 25 points or more on a scale of 30 points may be determined as registration. Evaluation standards may include accuracy of the information, professionalism, delivery power, and the like. Qualified holders of specialists in each department or higher can score points on a scale of 10 points for each evaluation standard. After the evaluation of a certain number of qualified holders is completed, the average of the evaluation scores is derived, and the video whose average score exceeds 25 points may be determined as registration.

According to one embodiment, the drug guidance video or health management video may be set to be viewed only by qualified holders of each department specialist or higher registered in the medical information provision server 100, and only the video for which registration has been decided may be registered in the video sharing platform 600.

The video-sharing platform 600 may be a social media platform such as YouTube, Facebook, and Instagram. The video-sharing platform 600 may share various media types, including text, image, audio, and video.

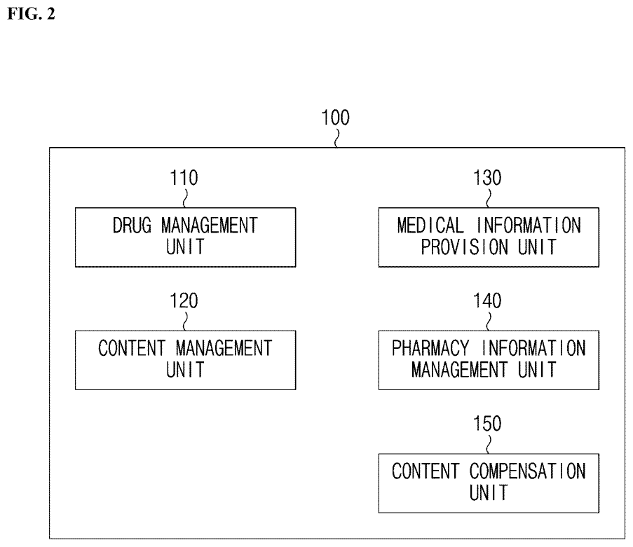

According to an embodiment, a drug guidance video or health management video determined to be registered by the medical information provision server 100 may be registered in the video-sharing platform 600. The detailed descriptions are describe d later with reference to FIGS. 2, 3, and 4.

FIG. 2 is a configuration diagram schematically illustrating a medical information provision server 100 according to an embodiment of the present disclosure.

Referring to FIG. 2, the medical information provision server 100 comprises a drug management unit 110, a content management unit 120, a medical information provision unit 130, a pharmacy information management unit 140, and a content compensation unit 150.

The drug management unit 110 may store a combined serial number by assigning a unique number for each attribute of drug and a unique number for each subject of drug and additional information related to a disease. For example, Tylenol™ is given a unique number for each attribute, such as 6 digits 112201 through antipyretic analgesic-11,acetaminophen-22, and 500 mg-01. Further, if the content is changed to 650 mg, although the efficacy and the same ingredient, the last two digits may be designated as 02. Further, additional unique numbers for gender and age are assigned to the above number so that a unique number for each attribute of drug and a unique number for each subject of drug may be given to store the combined serial number. For example, there are 112201-MO, 112201-FY, 112201-Y, and 112201-O in which serial numbers such as M=male, F=female, Y=young, and O=old may be assigned to each drug. In the case of age, for example, generation may be classified based on the age of 40, Y (young) may be assigned to those younger than 40 years old, and O (elderly) may be assigned to those over 60 years old.

According to one embodiment, additional unique numbers may be assigned for underlying diseases, allergies, and the like. AL may be assigned for allergies, and numbers may be assigned according to the type of allergy so that it may be configured to have a unique number such that AL01 may be assigned for drug allergies and AL02 may be assigned for food allergies. In the case of an underlying disease, the corresponding disease classification code may be assigned as a unique number. Therefore, the serial number for each drug is based on the combination of the unique number for each attribute of the drug and the unique number for each subject to be taken. However, the serial number may be created by giving an additional unique number for the underlying disease or allergy. Accordingly, when information such as an underlying disease or allergy is input by the user, the accuracy of customized medical information to be provided to the user may be further increased.

The content management unit 120 may provide the list by serial number to at least one video providing terminal 500, receive at least one drug guidance video or health management video corresponding to the serial number, and register the video to the video sharing platform 600 when the evaluation score is above the reference value. Specifically, the serial number list may be displayed on the video providing terminal 500 with the serial number and the name, efficacy, ingredient, and content of drug corresponding to the serial number. Further, video standard information including video time, quality, format, etc., may be additionally provided to secure video consistency. The obtained drug guidance video and health management video are evaluated, and an evaluation score is calculated. When the evaluation score is higher than the reference value, it is registered on the video-sharing platform, and incentives may be paid to the video provider. For example, the evaluation of a drug guidance video or health management video may be performed by qualified holders of a specialist or higher in each department pre-registered in the medical information provision server 100, and a video with a score of 25 points or more on a scale of 30 points may be determined as registration. Evaluation standards may include accuracy of the information, professional ism, delivery power, and the like. Qualified holders of specialists in each department or higher can score points on a scale of 10 points for each evaluation standard. After the evaluation of a certain number of qualified holders is completed, the average of the evaluation scores is derived, and the video whose average score exceeds 25 points may be determined as registration.

The content management unit 120 sets the title of the drug guidance video or health management video according to a predetermined rule based on the serial number, automatically creates subtitles for each language based on artificial intelligence, and automatically matches advertisements related to the drug guidance video or health management video.

More specifically, setting the title of a drug guidance video or health management video according to a certain rule based on the serial number is as follows. For example, Tylenol is assigned a drug serial number of 112201-MO, which is a combination of a 6-digit unique number for antipyretic analgesic-11, acetaminophen-22, and 500 mg-01 and a unique number for each subject. In this case, since there is information such as antipyretic analgesic, acetaminophen, 500 mg, male, elderly, titles such as “Elderly male's cold medicine taking guidance” or “Elderly male's cold prevention and treatment” are given to the video received with the serial number for each drug. Further, when a unique number for an underlying disease or allergy is added, titles such as Elderly male with diabetes's cold medicine taking guidance” or “Elderly male with diabetes's cold prevention and treatment” may be assigned.

Subtitles for each language may be generated through automatic translation after first converting the voice in the video into text using the AI-based speech-to-text method. The AI-based speech-to-text method may be implemented using open speech processing APIs such as Google Cloud Speech API, IBM Watson Speech to Text, Microsoft Azure Bing Speech API, and Amazon Transcribe. Automatic translation for each language may be implemented using commercial artificial intelligence automatic translation API services such as Google Translation API, Kakao Translation API, and Papago Translation API.

Further, an advertisement related to a cold of a diabetic elderly male may be matched based on the serial number for each drug, and the corresponding advertisement may be provided at the bottom of the video or added before or after the video.

The medical information provision unit 130 may learn drug-related diseases and health information related to the diseases, calculate the degree of relevance by drug serial number for drug guidance video, health management video, and additional information related to a disease and generate the customized medical information based on the degree of relevance between prescription information and each drug serial number.

Specifically, only one prescription drug may be provided for a simple disease such as a cold, but multiple drugs may be prescribed according to cold symptoms or a doctor's prescription, and various types of drugs may be prescribed according to the type of disease. Therefore, big data on prescriptions are collected, and preprocessing is performed on the big data on prescriptions first. The information included in the prescription includes insurance classification, personal information, hospital information, disease classification code, the doctor in charge information, drug name (efficacy and ingredient), single dose (content), number of administrations per day, total number of days of administration, usage, etc. Among them, personal information, disease classification code, drug name, single dose, number of administrations per day, the total number of days of the administration, usage, etc. may be labeled, and the relevance between disease classification code and personal information and at least one drug may be learned. Further, among personal information, sensitive information other than age and gender may be excluded. Further, age may be transformed into age group. For example, 32 years old is set as 30 years old group, and 56 years old is set as 50 years old group to prevent data fragmentation. Based on the preprocessed data, the degree of relevance for each drug serial number may be calculated according to the disease classification code, age, and gender through machine learning, and the accuracy of the machine learning algorithm may be corrected through a verification process.

The pharmacy information management unit 140 may manage information on registered pharmacies and provide a list of a plurality of nearby pharmacies to the user terminal 200 based on location information of the user terminal 200. Based on the the location information of the user terminal 200, a nearby pharmacy may be displayed on a map through a customized medical information application, or a list of pharmacies may be displayed in order of proximity.

The content compensation unit 150 may provide an incentive to the video-providing terminal 500 that has provided the drug guidance video or health management video registered in the video-sharing platform 600. Incentives may be provided in various forms, such as cash payment through an account, points, or coupons. Therefore, video providers may have the motivation to produce higher-quality content.

The attributes of the drug include efficacy, ingredients, and content, and the subject of the drug includes gender, age group, underlying disease, and allergy. The additional information related to a disease may be any one of text, image, audio, and video. For example, for diabetes, news related to causes, symptoms, treatment, prevention, diet and lifestyle, new drugs, or new treatments of diabetes may be provided in the form of text, image, audio, or video. If necessary, the latest information may be appropriately processed and provided in the form of card news.

According to an embodiment, the medical information provision server 100 may store the prescription information in a private blockchain. Prescription information is patients' sensitive information, so the risk of hacking can be prevented by storing it in the blockchain. To this end, it is possible to provide users with control over individual prescription information based on DID identity authentication (decentralized identity). If there is a user's consent, the medical information provision server 100 may use the prescription information and provide various customized medical information.

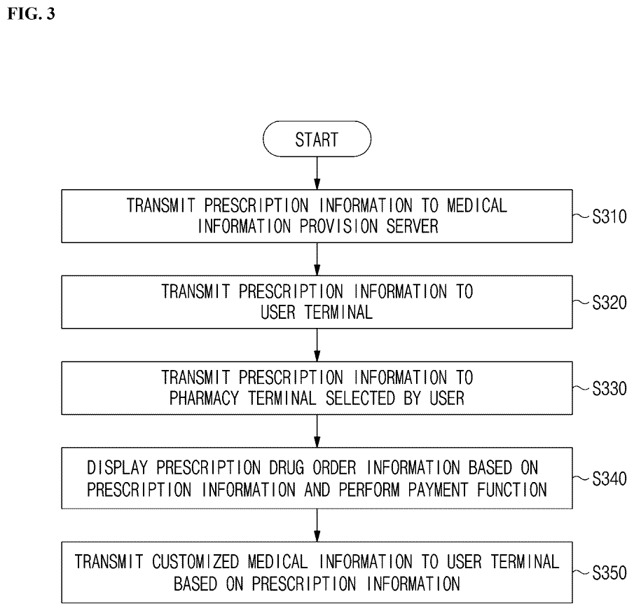

FIG. 3 is a flowchart illustrating an artificial intelligence-based customized medical information provision method according to an embodiment of the present disclosure.

Referring to FIG. 3, the method may comprise steps S310 of transmitting, by a hospital terminal 300, prescription information to a medical information provision server 100; S320 of transmitting, by the medical information provision server 100, the prescription information to a user terminal 200; S330 of transmitting, by the user terminal 200, the prescription information to a pharmacy terminal 400 selected by the user; S340 of displaying, by the pharmacy terminal 400, prescription drug order information based on the prescription information and performing a payment function; and S350 of transmitting, by the medical information provision server 100, customized medical information including at least one drug guidance video or health management video, and additional information related to a disease registered in a video sharing platform 600 based on the prescription information to the user terminal 200.

S310 of transmitting, by a hospital terminal 300, prescription information to a medical information provision server 100 may be configured to transmit automatically. When the doctor inputs the prescription information into the hospital terminal 300 to issue a prescription, the prescription information is sent to the medical information provision server 100 simultaneously. The prescription information includes insurance classification, personal information, hospital information, disease classification code, the doctor in charge information, drug name, single dose, number of administrations per day, the total number of days of the administration, and usage.

In S320 of transmitting, by the medical information provision server 100, the prescription information is sent to a user terminal 200. When the medical information provision server 100 receives the prescription information, it automatically transmits a push notification notifying that the user terminal 200 has received the prescription information, and the prescription information may be received and displayed through the user terminal 200. According to an embodiment, the user terminal 200 may receive the prescription information through a customized medical information application provided by the medical information provision server 100.

In S330 of transmitting, by the user terminal 200, the prescription information to a pharmacy terminal 400 selected by the user, the user terminal 200 may display nearby pharmacies on a map or display a list of pharmacies in order of local distance based on location information determined based on GPS or a mobile communication network. The prescription information is transmitted to the pharmacy terminal 400 of the selected pharmacy among them based on the user input.

In S340 of displaying, by the pharmacy terminal 400, prescription drug order information based on the prescription information and performing a payment function, the pharmacy terminal 400 receives the prescription information. It displays prescription drug order information, and the pharmacist may confirm receipt of the prescription information through a notification function or the like. Further, a message notifying reception of prescription information may be transmitted to a pharmacist's portable terminal (not shown) connected to the pharmacy terminal 400 through a communication network.

In S350 of transmitting, by the medical information provision server 100, customized medical information including at least one drug guidance video or health management video, and additional information related to a disease registered in a video sharing platform 600 based on the prescription information to the user terminal 200, the drug guidance video or health management video may be directly embedded and played in a customized medical information application. Further, ink information on the drug guidance video and health management video may be provided through a customized medical information application, messenger, or text message according to the selection by the user terminal 200. The link information of the drug guidance video or health management video indicates the address information of the drug guidance video or health management video registered in the video sharing platform 600.

S350 of transmitting, by the medical information provision server 100, customized medical information based on the prescription information to the user terminal 200 may further comprise the step of generating the customized medical information based on the relevance between the prescription information and each drug serial number (not shown). In other words, information such as disease classification code, age, drug name, single dose, number of administrations per day, the total number of days of the administration, and usage are extracted from prescription information, and the drug serial number most closely related to this is selected. A drug guidance video, health management video, or additional information related to a disease corresponding to the drug serial number may be generated as customized medical information. The customized medical information generated by the medical information provision server 100 may be provided through a push alarm or a medical information-providing application. Further, the customized medical information may be checked within the medical information-providing application. Further, it may be directly moved to the video-sharing platform 600 and checked according to the user's selection.

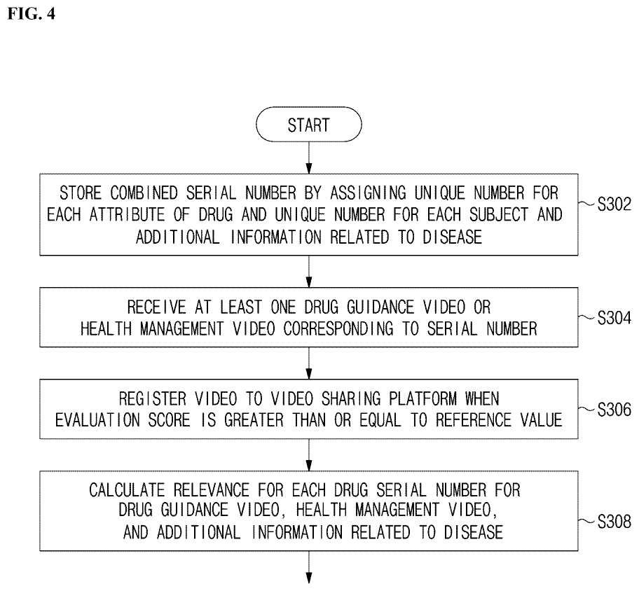

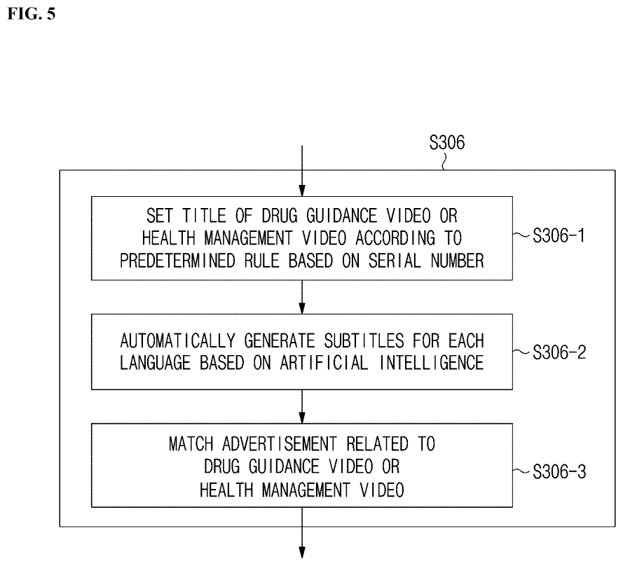

FIG. 4 is a flowchart illustrating a preparation process for generating customized medical information according to an embodiment of the present disclosure, and FIG. 5 is a flowchart illustrating a preprocessing process for a drug guidance video or health management video for registration the video to a video sharing platform 600 according to an embodiment of the present disclosure.

Referring to FIG. 4, the method may comprise, before S310 of transmitting prescription information to the medical information provision server 100, S302 of storing a combined serial number by assigning a unique number for each attribute of the drug and a unique number for each subject and additional information related to a disease; S304 of providing the list by serial number to at least one video providing terminal 500 and receiving at least one drug guidance video or health management video corresponding to the serial number; S306 of registering the video to the video sharing platform 600 when the at least one drug guidance video or health management video evaluation score is greater than or equal to a reference value; and S308 of learning a disease related to drug and health information related to the disease and calculating a degree of relevance for each drug serial number for drug guidance video, health management video, and additional information related to a disease.

According to one embodiment, the prescription information may include insurance classification, personal information, hospital information, disease classification code, the doctor in charge information, drug name, single dose, number of administrations per day, the total number of administration days, and usage, the attributes of the drug may include name, efficacy, ingredient, and content, the subject of use includes gender, age group, underlying disease, and allergy, and the additional information related to a disease may include any one of text, image, audio, and video.

In S302, storing a combined serial number by assigning a unique number for each attribute of the drug and a unique number for each subject and additional information related to a disease. For example, of a unique number for each drug attribute, Tylenol™ may be configured as 6 digits 112201 through antipyretic analgesic-11, acetaminophen-22, and 500 mg-01. Further, if the content is changed to 650 mg although the efficacy and the same ingredient, the last two digits may be designated as 02. Further, additional unique numbers for gender and age are assigned to the above number so that a unique number for each attribute of drug and a unique number for each subject of drug may be given to store the combined serial number. For example, there are 112201-MO, 112201-FY, 112201-Y, and 112201-O in which serial numbers such as M=male, F=female, Y=young, and O-old may be assigned to each drug. In the case of age, for example, generation may be classified based on the age of 40, Y (young) may be assigned to those younger than 40 years old, and O (elderly) may be assigned to those over 60 years old.

According to one embodiment, additional unique numbers may be assigned for underlying diseases, allergies, and the like. AL may be assigned for allergies, and numbers may be assigned according to the type of allergy so that it may be configured to have a unique number such that AL01 may be assigned for drug allergies, and AL02 may be assigned for food allergies. In the case of an underlying disease, the corresponding disease classification code may be assigned as a unique number. Therefore, the serial number for each drug is based on the combination of the unique number for each attribute of the drug and the unique number for each subject to be taken. However, the serial number may be created by giving an additional unique number for the underlying disease or allergy. Accordingly, when information such as an underlying disease or allergy is input by the user, the accuracy of customized medical information to be provided to the user may be further increased.

In S304 of providing the list by serial number to at least one video providing terminal 500 and receiving at least one drug guidance video or health management video corresponding to the serial number, the serial number list may be displayed on the video providing terminal 500 together with the serial number and corresponding attributes of the drug, such as name, efficacy, ingredient, and content. Further, video standard information, including video time, quality, format, etc., may be additionally provided to secure video consistency. The video providing terminal 500 may receive a list of combined serial numbers of a unique number for each property of drug and a unique number for each subject of drug assigned by the medical information provision server 100 and create and transmit at least one drug guidance video or health management video corresponding to the serial number to the medical information provision server 100. Preferably, students of medical school, pharmacy school, dental school, etc., which may be regarded as experts in the medical field, may provide drug guidance video or health management video through the video providing terminal 500 but are limited thereto. Those who can produce medical information-related videos may also provide a drug guidance video or health management video. This way, high-quality content can be supplied and supplied through the cloud-sourcing format for the serial number list provided by the medical information provision server.

Referring to FIGS. 4 and 5, S306 of registering the video to the video sharing platform 600 when at least one drug guidance video or health management video evaluation score is greater than or equal to a reference value may further comprise S306-1 of setting a title of a drug guidance video or health management video according to a predetermined rule based on the serial number; S306-2 of automatically generating subtitles for each language based on artificial intelligence; and S306-3 of matching an advertisement related to the drug guidance video or health management video.

In S306 of registering the video to the video sharing platform 600 when at least one drug guidance video or health management video evaluation score is greater than or equal to a reference, the drug guidance video or health management video received from the video providing terminal 500 is evaluated, and the evaluation score is calculated. The video may be registered on the video-sharing platform if the evaluation score exceeds the reference value. For example, the evaluation of a drug guidance video or health management video may be performed by qualified holders of a specialist or higher in each department pre-registered in the medical information provision server 100, and a video with a score of 25 points or more on a scale of 30 points may be determined as registration. Evaluation standards may include accuracy of the information, professionalism, delivery power, and the like. Qualified holders of specialists in each department or higher can score points on a scale of 10 points for each evaluation standard. After the evaluation of a certain number of qualified holders is completed, the average of the evaluation scores is derived, and the video whose average score exceeds 25 points may be determined as registration.

In S306-1, setting the title of a drug guidance video or health management video according to a certain rule based on the serial number is as follows. For example, Tylenol is assigned a drug serial number of 112201-MO, which is a combination of a 6-digit unique number for antipyretic analgesic-11, acetaminophen-22, and 500 mg-01 and a unique number for each subject. In this case, since there is information such as antipyretic analgesic, acetaminophen, 500 mg, male, elderly, titles such as “Elderly male's cold medicine taking guidance” or “Elderly male's cold prevention and treatment” are given to the video received with the serial number for each drug. Further, when a unique number for an underlying disease or allergy is added, titles such as Elderly male with diabetes's cold medicine taking guidance” or “Elderly male with diabetes's cold prevention and treatment” may be assigned.

For example, Tylenol is assigned a drug serial number of 112201-MO, which is a combination of a 6-digit unique number for antipyretic analgesic-11, acetaminophen-22, and 500 mg-01 and a unique number for each subject. In this case, since there is information such as antipyretic analgesic, acetaminophen, 500 mg, male, elderly, titles such as “Elderly male's cold medicine taking guidance” or “Elderly male's cold prevention and treatment” are given to the video received with the serial number for each drug. Further, when a unique number for an underlying disease or allergy is added, titles such as Elderly male with diabetes's cold medicine taking guidance” or “Elderly male with diabetes's cold prevention and treatment” may be assigned.

In S306-2, subtitles for each language may be generated through automatic translation after first converting the voice in the video into text using the AI-based speech-to-text method. The AI-based speech-to-text method may be implemented using open speech processing APIs such as Google Cloud Speech API, IBM Watson Speech to Text, Microsoft Azure Bing Speech API, and Amazon Transcribe. Automatic translation for each language may be implemented using commercial artificial intelligence automatic translation API services such as Google Translation API, Kakao Translation API, and Papago Translation API. Text may be generated from the audio of the received video by applying speech to text (STT) method, and subtitles for each language may be generated by applying artificial intelligence automatic translation technology to the text. Therefore, subtitle data for each language may be added to the video. Accordingly, although the user is not fluent in Korean, it is possible to obtain information more easily through subtitles for each language provided in the video.

In S306-3, an advertisement related to a cold of a diabetic elderly male may be matched based on the serial number for each drug, and the corresponding advertisement may be provided at the bottom of the video or added before or after the video. Among advertisements registered from various organizations or institutions, an advertisement with high relevance may be matched based on the serial number for each drug. For example, advertisements selected through keyword-based advertisement matching may be provided to videos. The advertisement may be provided in text or image at the bottom of the video or before or after the video.

In S308 of learning a disease related to drug and health information related to the disease and calculating a degree of relevance for each drug serial number for the drug guidance video, health management video, and additional information related to a disease, only one prescription drug may be provided for a simple disease such as a cold, but multiple drugs may be prescribed according to cold symptoms or a doctor's prescription, and various types of drugs may be prescribed according to the type of disease. Therefore, big data on prescriptions are collected, and preprocessing is performed on the big data on prescriptions first. The information included in the prescription includes insurance classification, personal information, hospital information, disease classification code, the doctor in charge information, drug name (efficacy and ingredient), single dose (content), number of administrations per day, the total number of days of administration, usage, etc. Among them, personal information, disease classification code, drug name, single dose, number of administrations per day, the total number of days of the administration, usage, etc., may be labeled, and the relevance between disease classification code and personal information and at least one drug may be learned. Further, among personal information, sensitive information other than age and gender may be excluded. In the case of age, for example, generation may be classified based on the age of 40, Y (young) may be assigned to those younger than 40 years old, and O (elderly) may be assigned to those over 60 years old. The degree of relevance for each drug serial number may be calculated according to the disease classification code, age, and gender through machine learning based on the preprocessed data, and the accuracy of the machine learning algorithm may be corrected through a verification process.

According to one embodiment, the method may further comprise, after S306 of registering the video to the video sharing platform 600 when at least one drug guidance video or health management video evaluation score is greater than or equal to a reference, providing an incentive to a video providing terminal 500 that provided a drug guidance video, or a health management video registered in the video sharing platform 600. For example, in S306, incentives may be provided in various forms, such as cash payment through an account, points, or coupons to the video provider of the video providing terminal 500 that provided a drug guidance video or health management video having an evaluation score higher than the reference value in various forms such as cash payment through an account, points, coupons, etc. Therefore, video providers may produce higher-quality content.

According to one embodiment, S340 of displaying, by the pharmacy terminal 400, prescription drug order information based on the prescription information and performing a payment function may further comprise the step of transmitting drug guidance information according to the pharmacist's input to the user terminal 200 (not shown). The drug guidance information may be information such as directions for prescription drugs which the pharmacist input through the pharmacy terminal 400. For example, drug guidance information such as 30 minutes after a meal or 30 minutes before a meal, 3 times a day, a list of foods to be careful of, possible side effects, and countermeasures during the intake period may be provided in the form of a pharmacist's voice or text. Further, the drug guidance information may be provided by converting it into text form through voice recognition of the pharmacist's voice or video.

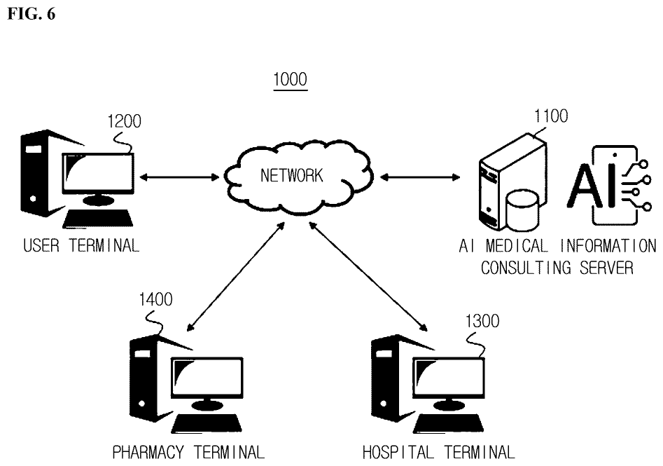

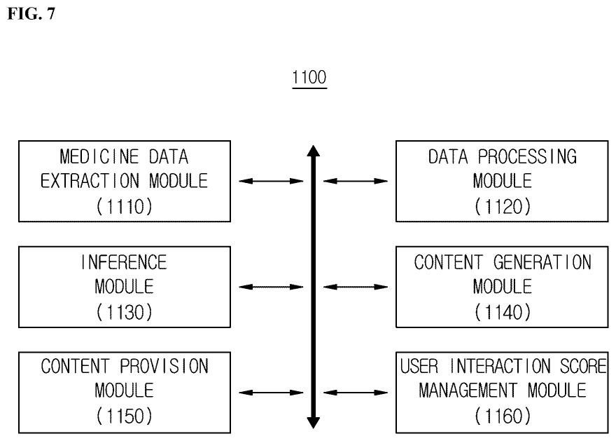

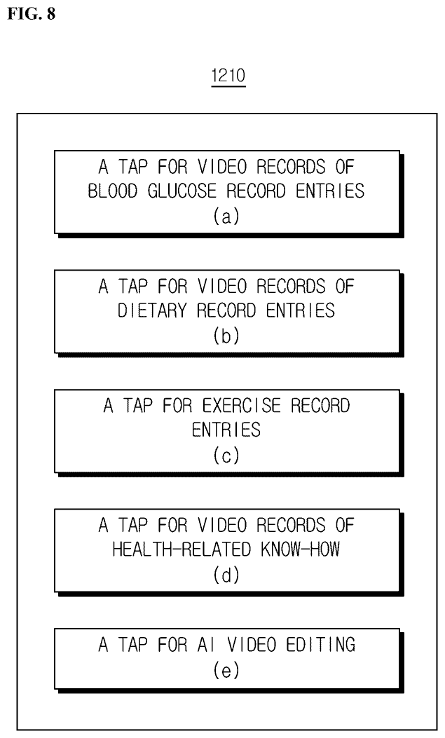

FIG. 6 is a configuration diagram schematically illustrating an AI-based personalized medical information provision system according to an embodiment of the present disclosure. FIG. 7 is a configuration diagram, depicting details of a server for an AI-based personalized medical information provision system shown in FIG. 6. FIG. 8 is an example diagram of the Open API functions running on the user terminals shown in FIG. 6.

Referring to FIGS. 6-8, an AI-based personalized medical information provision system 1000 according to an embodiment of the present disclosure may include the AI medical information consulting server 1100, the user terminal 1200, the hospital terminal 1300, and the pharmacy terminal 1400.

Each of its components is connected to networks. The network refers to a connection structure that enables information exchange between nodes such as a plurality of devices and servers. Examples of the network may include local area networks LAN, wide area networks WAN, world wide web WWW, wired/wireless data communication networks, telephone networks, wired/wireless television communication networks, and the like. Examples of the wireless data communication network include 3G, 4G, 5G, 3^{rd} generation partnership project 3GPP, 5^{th} generation partnership project 5GPP, long term evolution LTE, world interoperability for microwave access WIMAX, Wi-Fi, internet, local area network LAN, Wireless local area network, wide area network WAN, personal area network PAN, radio frequency RF, bluetooth networks, near-field communication networks, satellite broadcast networks, analog broadcast networks, analog broadcast networks, and the like, but are not limited thereto.

The user terminal 1200 may receive prescription information from the hospital terminal 1300 using the Open API 1210. The user terminal 1200 may receive prescription records from external servers such as the National Health Insurance Service Center after verifying the identity of the user.

Prescriptions may contain various information, including source of insurance, demographic information, hospital information, disease classification symbol, referring doctor, drug name, single dose, dosage per day, duration, and instructions for use.

In addition, the user terminal 1200 may use the Open API 1210 to transmit and receive a list of a plurality of nearby pharmacies based on location information, and may transmit prescription information to the pharmacy terminal 1400 corresponding to the pharmacy selected by the user according to a user selection.

By receiving prescription information from a hospital terminal through an Open API, data interoperability between medical institutions and systems can be enhanced. The Open API facilitates integration with various hospital systems, making system development and maintenance easier, while enabling the collection of comprehensive medical data.

It also provides the flexibility for integration with various platforms and applications, which is a significant advantage in transmitting medication guidance information or personalized healthcare content to a wide range of user devices. For example, it enables the delivery of the same medical services across multiple devices such as smartphones, tablets, and PCs.

Furthermore, by integrating diverse external data, the Open API allows for more personalized services. For instance, it can deliver content tailored to the user's age group, gender, and type of illness via SNS or other Open APIs, ensuring that users receive precisely the information they need.

The user terminal 1200 may be equipped with a program configured to obtain prescription information either through optical character recognition (OCR) of an image captured of a prescription provided by a hospital, or through recognition of a QR code provided within the prescription.

In addition, the user terminal 1200 may automatically search highly relevant video content as users enter their age and gender by analyzing age, gender and picture captured by a camera after making a video record of a screen of a blood glucose monitoring device or a heart rate monitoring device using a camera function of an Open API, allowing users to check their health status and review the products or services related to their illness provided with video advertisements.

As shown in FIG. 8, the Open API 1210 executed on the user terminal 1200 may support the following features: (a) a tap for video records of blood glucose record entries; (b) a tap for video records of dietary record entries; (c) a tap for exercise record entries; (d) a tap for video records of health-related know-how; and (e) a tap for AI video editing.

A tap for video records of blood glucose record entries (a) may be a function for recording the process of measuring blood glucose levels using the timer.

A tap for video records of dietary record entries (b) may be a function for recording the eating procedure using the timer.

A tap for exercise record entries (c) may be a function for recording what types of exercise a user is doing using the timer.

A tap for video records of health-related know-how (d) may be a function for recording a personal story for other patients and the recovery stories or the processes of recovering from illness using the timer.

A tap for AI video editing (e) may be a function of automatically editing all videos mentioned above into a single video using AI.

As shown in FIG. 6, the AI medical information consulting server 1100 provides a list of a plurality of nearby pharmacies based on location information of the user terminal 1200 using the Open API installed on the user terminal 1200.

The hospital terminal 1300 may be a terminal that inputs prescription information provided by the user terminal 1200. The hospital terminal 1300 can be configured to automatically transmit prescription information to the AI medical information consulting server 1100 once the prescription information is input.

The hospital terminal 1300 may request registration of advertisements for medical products provided by hospitals to the AI medical information consulting server 1100. For example, advertisements for health functional foods, supplements, medical assistive devices and the like that offer potential benefits related to a user's disease may be registered on the AI medical information consulting server 1100.

The pharmacy terminal 1400 may be a terminal that displays the prescription order based on prescription information provided by the user terminal and performs a payment function

The pharmacy terminal 1400 may request registration of advertisements for pharmacies, various products, and the like to the AI medical information consulting server 1100. For example, in a similar manner to the hospital terminal, it can make advertisements and request registration of the advertisements for medical information such as health functional foods or supplements that match a user's disease type.

The pharmacy terminal 1400 may transmit drug guidance information entered based on the prescriptions to the user terminal 1200 where the instructions for drug guidance are simply entered by a pharmacist through the pharmacist terminal 1400. For instance, information on drug guidance information may be provided in textual or spoken forms by a pharmacist, including a list of foods to be avoided during a medication period, 30 minutes before or after a meal, 3 times a day, potential side effects and countermeasures.

In addition, drug guidance information may contain information such as drug name (drug code), strength, dosage, duration, and so on. For instance, codes for classification of drugs are regulated by the Ministry of Food and Drug Safety in South Korea, but hospitals and insurance companies can use their own codes internally. For example, a drug code of the Ministry of Food and Drug Safety in Korea is a 10-digit number, containing a 5-digit labeler code, a 3-digit product code, and a last 2-digit formulation/dose code. (e.g. drug code: 12345-678-90-> labeler 12345/product 678/formulation/dose 90).

By sending prescription information to the pharmacy terminal 1400 selected based on the location of the user terminal 1200 in advance, users can select the pharmacy they want to visit and immediately send their prescriptions, without having to wait for prescriptions after their medical treatments, thereby can save time when picking up their prescriptions upon arrival at the pharmacy. Furthermore, users can check the instructions for use or precautions at any time by receiving drug guidance information entered by a pharmacist, which helps a user to take drugs the right way according to a medicinal usage.

As shown in FIG. 7, the AI medical information consulting server 1100 comprises a drug data extraction module 1110, a data processing module 1120, an inference module 1130, a content generation module 1140, a content provision module 1150, and a user engagement score management module 1160.

The drug data extraction module 1110 extracts structured drug data such as the drug type, dosage, administration frequency, and prescription period from drug guidance information.

The drug data extraction module 1110 extracts information such as the drug type, dosage, administration frequency, and prescription period from drug guidance information to create the structured data. The drug data extraction module 1110 extracts key drug-related information from prescriptions and organizes it systematically.

The data processing module 1120 preprocesses the extracted, structured drug data, and extracts the features of the data.

The data processing module 1120 preprocesses the structured drug data extracted from drug guidance information to be prepared in a format suitable for analysis, and to extract the major features of the data prepared via feature extraction. The prepared data can be used in the inference module 1130 to infer a disease type, a disease severity, and a physical safety status of the user.

The inference module 1130 applies the pre-trained machine learning model stored in the memory to drug data of the extracted preprocessing and features to infer a disease type, a disease severity, and a physical safety status of the user. The inference module 1130 predicts and evaluates the health status of the user from various angles based on the various data.

The machine learning model is trained based on a learning data set containing user's past prescription data, user health profiles, and treatment outcomes, and can be continuously updated based on user feedback or result validation.

More specifically, the machine learning model trains the model based on data, including a user's past prescription data, user health profiles, and treatment outcomes, and predicts a disease type, a disease severity, and a physical safety status of the user via extracting features and preprocessing of the structured drug data (e.g. drug type, dosage, administration frequency and prescription period). Furthermore, the machine learning model evaluates whether the user's health condition has improved, deteriorated, or remained stable using a time series analysis and analyzes the complications or complex diseases of the user using the rule-based inference engine.

By applying a pre-trained machine learning model to infer the user's type of illness, severity of the condition, and physical safety status, the system can accurately identify each user's medical profile. The AI medical information consulting server can use datasets containing the types of medications previously taken by the user, dosage, frequency, and prescription duration to allow the model to estimate how severe the illness is. This enables users to establish more appropriate treatment plans.

Additionally, the machine learning model can evaluate the user's physical safety status based on physiological information, helping determine whether the current medication can be taken in combination with other drugs or detecting potential side effects in advance.

Moreover, based on the inferred results, the machine learning model can provide personalized healthcare content. For example, the AI medical information consulting server may generate and deliver content on precautions, activities to avoid, and proper medication methods, contributing to the user's overall health management.

The machine learning model can also predict the progression of a disease based on user data, enabling early intervention for disease prevention. In the long term, this helps users achieve safer and more effective medication use and health management.

As an example, the machine learning model can classify various types of diseases of users into categories based on various sources of medical data and user profiles when inferring a disease type, a disease severity, and a physical safety status of the user using a classification model.

As an example, the machine learning model evaluates whether the user's health condition has improved, deteriorated, or remained stable based on the prescription or evaluates disease severity using a regression module. Furthermore, the machine learning model provides the personalized content by grouping the users based on their age, gender, activity levels, and so on using a clustering model, and compares/analyzes the participation level.

The machine learning model is merely an example and other and/or additional types of models can be used. The machine learning model plays a key role in providing medical information and personalized services to users, and each model can be configured to provide medical solutions using data such as the user behavior data and medical data.

In addition, the machine learning model can continuously update the model based on user feedback or result validation to improve accuracy. The system for providing AI-based personalized medical information of the present disclosure can generate personalized healthcare content, recommendations, and alerts to deliver them to the users.

The content generation module 1140 generates personalized healthcare content, including alerts, recommendations, and health tracking prompts base on the inference results.

Health tracking prompts are notifications or messages that continuously monitor a user's health related information and provide appropriate alerts, recommendations, and healthcare directives in real-time. Health tracking prompts may include alerts provided in situations where a user's condition requires immediate attention, directives related to health indicators or activities that should be tracked on a regular basis by a user, or personalized healthcare content based on the user's past health-related records and current condition.

The content provision module 1150 transmits the generated, personalized healthcare content to the user terminal in real-time via a secured communication channel.

The user engagement score management module 1160 monitors the participation level of the user by analyzing dietary patterns and exercise records received from the user terminal, and generates the user engagement scores by comparing the participation level with those of other users within the user cluster based on age and gender.

The content provision module 1150 of the AI medical information consulting server 1100 can search a plurality of pre-stored drug guidance video contents, and content corresponding to an age group or gender of the user to transmit the selected content to the user terminal via an Open API or social networking service.

In addition, the content provision module 1150 of the AI medical information consulting server 1100 may be configured to select the personalized video content for patients based on the viewing frequency of videos enjoyed by patients with similar symptoms to provide them to the user terminal via an Open API or social networking service.

In addition, the content provision module 1150 of the AI medical information consulting server 1100 may be configured to select a video related to alerts of serious side effects from the beginning of health education on diseases to provide them to the user terminal via an Open API or social networking service.

In addition, the content provision module 1150 of the AI medical information consulting server 1100 may be configured to provide videos that help improve lifestyle habits to prevent disease and educational videos on the historical, cultural, and ethnological meanings of illness.

In addition, the inference module 1130 of the AI medical information consulting server 1100 can evaluate a severity level of a disease based on at least one of a drug type, dosage, administration frequency, and prescription period, and analyze whether the user has a comorbidity or complication based on user physical information and information regarding concurrently administered drugs through a rule-based inference engine.

In addition, the inference module 1130 of the AI medical information consulting server 1100 can analyze the user's past prescription history in chronological order, and evaluates whether the user's health condition has improved, deteriorated, or remained stable using a time series analysis using a time series analysis.

In addition, the inference module 1130 of the AI medical information consulting server 1100 may monitor a viewing history of video content played on the user terminal, and run the behavioral analysis algorithm that measures the treatment adherence level by comparing the monitored viewing frequency with an average viewing frequency of other users.

In addition, the inference module 1130 of the AI medical information consulting server 1100 can provide content to users by adjusting an exposure frequency of warning video content based on the evaluation of treatment adherence via an optimization module.

In addition, the AI medical information consulting server 1100 can further include the user engagement score management module 1160 that monitors the participation level of users by analyzing dietary patterns and exercise records received from the user terminal, and generates the user engagement scores by comparing the participation level with those of other users within the user cluster based on age and gender.

In addition, the user engagement score management module 1160 of the AI medical information consulting server 1100 can calculate the accumulated points based on at least one of the user's content viewing frequency, the number of blood glucose record entries, the number of dietary record entries, and the number of exercise record entries, and store the accumulated points in a point management table for each user.

In addition, the user engagement score management module 1160 of the AI medical information consulting server 1100 can assign a user ranking depending on the user engagement scores in the user cluster, and calculate real-time user ranks based on the degree of disease improvement, the level of health achievement, and the duration of health maintenance.

In addition, the user engagement score management module 1160 of the AI medical information consulting server 1100 may adjust a real-time grades of a user based on changes in prescription information of a user (e.g. strength, dosage, duration).

In addition, the user engagement score management module 1160 of the AI medical information consulting server 1100 may provide the user interface UI for preparing health-related know-how content to the user terminal having a grade equal to or higher than a predetermined threshold; assign the pre-defined reward points based on a viewing frequency of the content created by the corresponding user terminal; and record a transaction history of the reward points in storage devices.

When the user's viewing frequency of health-related know-how content meets certain criteria, points can be awarded to the users based on predetermined criteria. The rewards transaction history and accumulated points will be stored in blockchain-based storage, ensuring reliability and security as they are recorded in a transparent and immutable manner, leveraging the characteristics of blockchain, ensuring reliability and security. The present disclosure encourages users to participate in creating video content, transparently manages rewards for health-related activities, and utilizes blockchain to ensure security and data immutability.

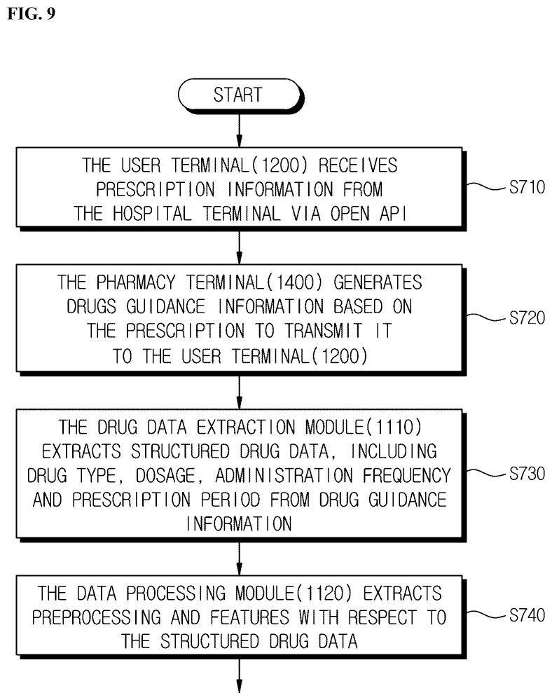

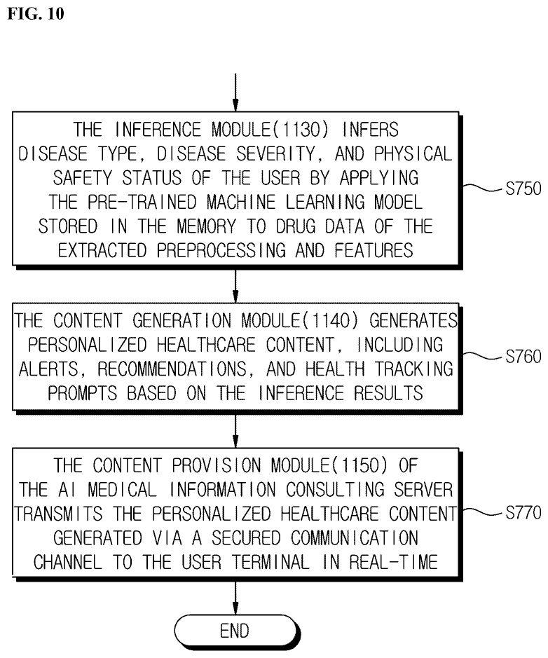

FIG. 9 and FIG. 10 are flowcharts illustrating a method for providing AI-based personalized medical information according to an embodiment of the present disclosure.

The method for providing AI-based personalized medical information according to an embodiment of the present disclosure (S700) includes steps of collecting prescription information provided by the hospital terminal 1300, drug guidance information provided by the pharmacy terminal 1400, and patient information provided by the user terminal 1200 in the AI medical information consulting server 1100; evaluating a patient's disease using the collected data; and providing personalized healthcare content based on the evaluation results.

Hereinafter, the method for providing AI-based personalized medical information (S700) is explained in more detail.

First, the step in which the user terminal 1200 receives prescriptions from the hospital terminal via Open API (S710) is executed.

Next, the step in which the pharmacy terminal 1400 generates drug guidance information based on the prescription and transmits it to the user terminal 1200 (S720) is executed.

The step in which the drug data extraction module 1110 of the AI medical information consulting server extracts the structured drug data, including the drug type, dosage, administration frequency and prescription period from drug guidance information (S730) is executed.

The drug data extraction module 1110 may analyze the drug name (drug code) within drug guidance information generated by the pharmacy terminal 1400. The drug data extraction module 1110 may analyze the drug name (drug code) by classifying it into segments: labeler code, product code, formulation code, dosage code.

The drug data extraction module 1110 may analyze raw data of country-specific or hospital/institution-specific drug codes.

The drug data extraction module 1110 may classify codes by using a parsing process when analyzing drug codes. Code classification cuts the codes into segments or divides them with delimiters to analyze or assigns unique identifiers to medications based on certain rules.

After classifying the drug codes, the drug data extraction module 1110 may search and match the actual drug name, strength, efficacy, and so on by code using standard data of Health Insurance Review & Assessment Service.

Next, the step in which the data processing module 1120 of the AI medical information consulting server extracts preprocessing and features with respect to the structured drug data (S740) is executed.

Next, the step in which the inference module 1130 of the AI medical information consulting server infers a disease type, a disease severity, and a physical safety status of the user by applying the pre-trained machine learning model stored in the memory to drug data of the extracted preprocessing and features (S750) is executed. The machine learning model of the method for providing AI-based personalized medical information can be trained based on the training dataset, including past prescription data, health profiles of a user, and treatment outcomes, and continuously updated based on user feedback or result validation.

The inference module 1130 may infer a disease type, a disease severity, and a physical safety status of the user based on the structured drug data, including the drug type, dosage, administration frequency and prescription period from the drug data extraction module 1110 (S750).

More specifically, the inference module 1130 can include a process of inferring a patient's disease using information on illness/indications.

When there are multiple indications, the inference module 1130 can set priorities. Priority criteria can be set based on the number of insurance claims, main implications, and so on.

The inference module 1130 may include a process of inputting a list of drugs a patient is currently taking into the ingredient-disease table and classify patients according to their diseases through duplicate removal and multi-disease classification when the multiple drugs overlap. Additionally, the inference module 1130 uses the drug codes and diagnosis codes to classify patient illness.

The inference module 1130 may evaluate a severity level of a disease using structured data, including at least one of the drug type, dosage, administration frequency, and presecription period. The inference module 1130 can apply the machine learning model to evaluate a severity level of a disease based on data of age/gender, drug name/efficacy/dosage, comorbidity according to the type and quantity of drugs concurrently taking, dosage per day, and the past prescription history.

When a disease type, a disease severity, and a physical safety status of the user are inferred, a step in which the content generation module 1140 generates personalized healthcare content, including alerts, recommendations, and health tracking prompts based on the inference results (S760) is executed.

The step in which the content provision module 1150 of the AI medical information consulting server transmits the personalized healthcare content generated via a secured communication channel to the user terminal in real-time (S770) is executed.

In step S770, the content provision module 1150 searches a plurality of pre-stored drug guidance video contents, and selects contents corresponding to an age group or gender of the user to transmit the selected content to the user terminal via an Open API or social networking service.

In addition, the step S770 may further include the step of transmitting videos that help improve lifestyle habits to prevent disease and educational videos on the historical, cultural, and ethnological meanings of illness to the user terminal via an Open API or social networking service.

In addition, a step of inferring a disease type, a disease severity, and a physical safety status of the user evaluates a severity level of a disease based on at least one of a drug type, dosage, administration frequency, and prescription period, and analyze, through a rule-based inference engine, whether the user has a comorbidity or complication based on user physical information and information regarding concurrently administered drugs.

In addition, the step of inferring a disease type, a disease severity, and a physical safety status of the user analyzes the users past prescription data in chronological order, and evaluates whether the user's health condition has improved, deteriorated, or remained stable using a time series analysis.

In addition, the step of inferring a disease type, a disease severity, and a physical safety status of the user monitors a viewing history of video content played on the user terminal, and runs the behavioral analysis algorithm that measures the treatment adherence level by comparing the monitored viewing frequency with an average viewing frequency of other users.

In addition, the method for providing AI-based personalized medical information according to an embodiment of the present disclosure (S700) can further include the step of monitoring the participation level of users by analyzing dietary patterns and exercise records received from the user terminal, and generating the user engagement scores by comparing the participation level with those of other users within the user cluster based on age and gender.

Thus, according to an embodiment of the present disclosure, personalized medial information can be provided only for the patient based on the patient's prescription records.

In addition, according to an embodiment of the present disclosure, it offers a precise understanding of a user's disease and prescription and support a user to actively take suitable actions for his/her own health.

Further, service providers may obtain various medical information through the collection of prescription information to provide higher quality customized medical information, and hospitals, pharmacies, and other related organizations registered in the service may be linked to create synergistic effects

In the above, preferred embodiments according to the present disclosure have been shown and described. However, the present disclosure is not limited to the above-described embodiments, and various modifications can be made by anyone having ordinary knowledge in the technical field to which the present disclosure belongs without departing from the gist of the present disclosure appended within the scope of the claims.

### DESCRIPTION OF REFERENCE NUMERALS

| | |
|---|---|
| 100: medical information | 110: drug management unit |
| provision server | |
| 120: content management unit | 130: medical information |
| | provision unit |
| 140: pharmacy information | 150: content compensation unit |
| management unit | |
| 200: user terminal | 300: hospital terminal |
| 400: pharmacy terminal | 500: video providing terminal |
| 600: video sharing platform | |
| 1100: AI medical information | |
| consulting server | |
| 1110: drug data extraction module | |
| 1120: data processing module | |
| 1130: inference module | |
| 1140: content generation module | |
| 1150: content provision module | |
| 1160: user engagement score | |
| management module | |
| 1200: user terminal | |
| 1210: Open API | |
| 1300: hospital terminal | |
| 1400: pharmacy terminal | |

What is claimed is:

1. A system for AI-based personalized medical information provision comprising at least one processor and a memory, comprising:
a user terminal configured to receive prescription information from a hospital terminal through an Open API;
a pharmacy terminal configured to generate drug guidance information based on the prescription data and to transmit drug guidance information to the user terminal; and
an AI medical information consulting server comprising the processor and the memory storing instructions that, when executed by the processor, causing the server to operate:
a drug data extraction module configured to extract structured drug data, including a drug type, dosage, administration frequency, and prescription period from the drug guidance information;
a data processing module configured to perform preprocessing and feature extraction on the structured drug data;
an inference module configured to apply a pre-trained machine learning model stored in the memory to the preprocessed and feature-extracted drug data to infer a disease type, a disease severity, and a physical safety status of a user;
a content generation module configured to generate personalized healthcare content including alerts, recommendations, and health tracking prompts based on the inference result; and
a content provision module configured to transmit the generated personalized healthcare content to the user terminal in real time through a secure communication channel;
wherein the machine learning model is trained based on training dataset, including past prescription data, health profiles of a user, and treatment outcomes, and is continuously updated based on the result validation or user feedback.

2. The system for AI-based personalized medical information provision system of claim 1, wherein the content provision module of the AI medical information consulting server is configured to:
search a plurality of pre-stored drug guidance video contents;
select content corresponding to an age group or gender of the user; and
transmit the selected content to the user terminal via a social networking service (SNS) or an Open application programming interface (API).

3. The system for AI-based personalized medical information provision system of claim 1, wherein the inference module of the AI medical information consulting server is configured to:
evaluate a severity level of a disease based on at least one of a drug type, dosage, administration frequency, and prescription period; and
analyze, through a rule-based inference engine, whether the user has a comorbidity or complication based on user physical information and information regarding concurrently administered drugs.

4. The system for AI-based personalized medical information provision system of claim 1, wherein the inference module of the AI medical information consulting server is configured to:
analyze a user's past prescription history in chronological order; and
evaluate whether the user's health condition has improved, deteriorated, or remained stable by applying a time-series analysis model.

5. The system for AI-based personalized medical information provision system of claim 1, wherein the inference module of the AI medical information consulting server is configured to execute a behavioral analysis algorithm that:
monitors a viewing history of content executed on the user terminal; and
evaluates a treatment adherence level by comparing the monitored viewing frequency with an average viewing frequency of other users.

6. The system for AI-based personalized medical information provision system of claim 5, wherein the content provision module of the AI medical information consulting server is configured to adjust an exposure frequency of warning content according to an evaluation of treatment adherence, and to provide the adjusted content to the user.

7. The system for AI-based personalized medical information provision system of claim 1,
wherein the AI medical information consulting server further comprises a user engagement score management module configured to analyze exercise records and dietary improvement records received from the user terminal, monitor a participation level of the user, compare the participation level with those of other users within a user cluster based on age and gender, and generate a user engagement score.

8. The system for AI-based personalized medical information provision system of claim 7,
wherein the user engagement score management module is configured to calculate accumulated points based on at least one of the user's content viewing frequency, the number of blood glucose record entries, the number of dietary record entries, and the number of exercise record entries, and to store the accumulated points in a point management table for each user.

9. The system for AI-based personalized medical information provision system of claim 7,
wherein the user engagement score management module is further configured to assign a user ranking within the user cluster based on the user's engagement score, and to calculate a real-time grade of the user according to at least one of the degree of disease improvement, the level of health achievement, and the duration of health maintenance.

10. The system for AI-based personalized medical information provision system of claim 9,
wherein the user engagement score management module is further configured to adjust the user's real-time ranking based on a change history of the user's prescription information, including at least one of dosage, administration frequency, and prescription period.

11. The system for AI-based personalized medical information provision system of claim 10,
wherein the user engagement score management module is further configured to:
provide a user interface (UI) for generating health know-how content to user terminals having a grade equal to or higher than a predetermined threshold;
award pre-defined reward points based on a viewing frequency of the content created by the corresponding user terminal; and
record a transaction history of the reward points in a blockchain-based storage.

12. A method of providing AI-based personalized medical information to a user, comprising:
receiving, by a user terminal via an Open API, prescription information from a hospital terminal;
generating, by a pharmacy terminal, drug guidance information based on the prescription information and transmitting the drug guidance information to the user terminal;
extracting, by a drug data extraction module of the AI medical information consulting server, structured drug data including a drug type, dosage, administration frequency and prescription period from the drug guidance information;
performing, by a data processing module of the AI medical information consulting server, preprocessing and feature extraction on the structured drug data;
inferring, by an inference module of the AI medical information consulting server, a disease type, disease severity, and physical safety status of the user by applying a pre-trained machine learning model stored in the memory to the preprocessed and feature-extracted drug data;
generating, by a content generation module of the AI medical information consulting server, personalized healthcare content including alerts, recommendations, and health tracking prompts based on the inference result; and
transmitting, by a content provision module of the AI medical information consulting server, the generated personalized healthcare content to the user terminal in real time through a secure communication channel,
wherein the machine learning model is trained based on a training dataset including past prescription data, user health profiles, and treatment outcomes, and is continuously updated based on user feedback or result verification.

13. The method of providing AI-based personalized medical information to a user of claim 12,
wherein the step of transmitting the generated personalized healthcare content to the user terminal in real time comprises:
searching a plurality of pre-stored medication guidance video contents;
selecting content corresponding to an age group or gender of the user; and
transmitting the selected content to the user terminal via an SNS or an Open API.

14. The method of providing AI-based personalized medical information to a user of claim 12,
wherein the step of inferring the user's disease type, disease severity, and physical safety status comprises:
evaluating the severity of the disease according to a drug type, dosage, administration frequency and prescription period; and
analyzing, through a rule-based inference engine, whether the user has a comorbid condition or complication based on the user's physical information and information on concurrently administered medications.

15. The method of providing AI-based personalized medical information to a user of claim 12,
wherein the step of inferring the user's disease type, disease severity, and physical safety status comprises:
analyzing the user's past prescription history in chronological order; and
evaluating, through a time-series analysis model, whether the user's health condition has improved, deteriorated, or remained stable.

16. The method of providing AI-based personalized medical information to a user of claim 12,
wherein the step of inferring the user's disease type, disease severity, and physical safety status comprises:
monitoring the viewing history of content executed on the user terminal; and
evaluating, through a behavioral analysis algorithm, the user's treatment adherence by comparing the viewing frequency with an average viewing frequency of other users.

17. The method of providing AI-based personalized medical information to a user of claim 12, further comprising:
analyzing exercise records and dietary improvement records received from the user terminal;
monitoring the user's participation level; and
generating a user participation score by comparing the user's participation level within a user cluster based on age and gender.
